(19) United States
(12) Patent Application Publication
(10) Pub. No.: US 2012/0021441 A1
(43) Pub. Date: Jan. 26, 2012

(54) IN-PROCESS CONTROL IN A METHOD FOR PRODUCING EPO

(75) Inventors: Franz-Rudolf Kunz, Gelnhausen (DE); Florian Glaser, Nombris (DE); Wolfgang Wienand, Bergisch Gladbach (DE); Rudolf Hanko, Dusseldorf (DE); Wilfried Eul, Alzenau (DE); Dietmar Reichert, Eschau (DE)

(73) Assignee: Evonik Degussa GmbH, Essen (DE)

(21) Appl. No.: 13/140,190

(22) PCT Filed: Dec. 7, 2009
(86) PCT No.: PCT/EP2009/066517

§ 371 Date: Oct. 13, 2011

(30) Foreign Application Priority Data
Dec. 16, 2008 (DE) 102008054716.6

(51) Int. Cl. G01N 33/74; G01N 21/78

(52) U.S. Cl. 435/7.92

(57) ABSTRACT

The invention relates to a method for determining the isoform composition of erythropoietin, comprising the following steps:
a) isoelectrical focusing of a sample comprising erythropoietin in a gel over a pH range having a lower limit of 2.5 to 3.5 and an upper limit of 5 to 8, wherein the sample comprising erythropoietin originates from a culture supernatant of erythropoietin producing eukaryotic cells;
b) transferring of the proteins comprised and separated in the gel to a membrane;
c) verifying the erythropoietin bound to the membrane by specific antibodies;
   and to a method for in-process control of culture supernatants of erythropoietin producing eukaryotic cells during the fermentative production process.

The present invention relates to a method for the detection of erythropoietin, especially for the in-process control of culture supernatants of erythropoietin-producing eukaryotic cells during the fermentative production process. The process is especially characterized in that the isoform composition of the produced erythropoietin can be directly determined with the help of a special method of isoelectrical focusing (IEF). Together with the data obtained from the determination of the erythropoietin content (preferably by means of ELISA) the quality of the synthesized raw product can already be evaluated during or directly after the fermentation process and thus the subsequent purification process can be controlled. This is especially advantageous when perfusion reactors are used.

Erythropoietin, abbreviated EPO, is a glycoprotein with a molecular weight of about 34 to 39 kDa. It consists of an unbranched polypeptide chain with 165 amino acids and an O-glycosidically bound (Ser 126) and three N-glycosidically bound (Asn 24, Asn 38, and Asn 83) sugar side chains (carbohydrate portion). The side chains consist of the monosaccharides mannose, galactose, fucose, N-acetylglycosamine, N-acetylgalactosamine and N-acetylneuraminic acid.

Erythropoietin can occur in different isoforms. This variance of the molecular weight of erythropoietin is due to the heterogeneity of the sugar chains which are terminally linked with neuraminic acid derivates. By means of different lengths and branches of the chains a variety of “sugar branches” can be constructed which result in the characterisation of all isoforms of an EPO molecule.

EPO is mainly produced in the kidneys and, as a growth factor, stimulates the formation of erythrocytes in the bone marrow. In case of renal failure the damaged kidneys do not produce enough or any EPO at all whereby not enough erythrocytes are derived from the stem cells of the bone marrow. This renal anaemia can be treated by administering physiological amounts of EPO which stimulate the formation of erythrocytes in the bone marrow. The EPO used for administration can either be obtained from human urine or can be generated by means of methods of gene technology. Since EPO is contained in the human body only in very small traces the isolation of EPO out of its natural source is practically impossible for therapeutic purpose. Consequently methods of gene technology offer the only economic possibility of producing this substance in higher amounts.

The recombinant production of erythropoietin by means of methods of gene technology mainly takes place in so-called CHO cells (Chinese Hamster Ovary) and basically three different methods are used for the cultivation of the eukaryotic host cells (described amongst others in EP-A-0 148 605 and EP-A-205 564; BioProcess International 2004, 46; Gorenflo et al., Biotech. Bioeng. 2002, 80, 438 and WO9501214).

In a batch process the medium and the cells are introduced into the bioreactor at the beginning of the cultivation. Until the cultivation has been completed neither nutrients are added nor cells are removed from the fermenter, only oxygen is added. When one or more substrates are consumed the process is terminated and the products are harvested from the fermentation supernatant.

The second known cultivation process is the continuous process during which fresh medium is continuously fed into the reactor and the product is removed from the fermenter accordingly. This leads to a continuous supply of nutrients whereas at the same time undesired metabolites such as the growth-inhibiting substances ammonium and lactate are removed or diluted. Thus, with the help of this process higher cell densities can be achieved and maintained over a comparably long period of time. So-called dialysis reactors enable a special case of the continuous process which makes possible that high-molecular substances such as proteins are kept in the fermenter whereas low-molecular substances such as substrates can be added or the main by-products ammonium and lactate can be removed from the system. The use of perfusion reactors for the microbial production of chemical compounds and proteins with different cell-retaining systems are also general knowledge and have also been described for the case of EPO.

Finally, the third possible process is the fed-batch fermentation where the cultivation is started with a fractional amount of the whole fermenter volume and after a short growth period fresh substrate is added. This makes higher cell densities and longer process periods possible compared to the batch process. Another advantage of this process is the fact that the metabolism of the cells can be influenced by the extent of feeding which can lead to a lower production of waste substances. Compared to the continuous process the product of the cells is accumulated in the fermenter over a longer period of time and so higher product concentrations are achieved which makes the subsequent working-up easier.

Extensive chromatographic purification processes are coupled to the fermentative process so that an EPO can be isolated from the supernatants which can be therapeutically used and which corresponds to the standard defined by the European Pharmacopoeia (Ph. Eur.; 01/2002:1316) or the Guidance on Biosimilar Medicinal Products Containing Recombinant Erythropoietins (EMEA/CHMP/94256/2005). In this regard the state of the art is described in a variety of processes such as in WO-A-05/121173, EP-A-0 228 452, EP-A-0 267 678, EP-A-0 830 376, EP-A-1 127 063, WO-A-03/045996, EP-A-0 428 267 and WO2005121173.

According to the processes disclosed in the state of the art an erythropoietin-comprising sample is subjected to a separation process in a polyacrylamide gel, for example isoelectrical focusing, and the proteins contained in the sample are separated by applying an electric field. The electrophoresis is followed by a so-called immunoblot (immuno-print or immuno-transfer) during which the proteins are transferred onto a membrane. Thus, a copy of the individual proteins is obtained on the surface of the membrane. The membranes used have the advantage that the proteins are strongly fixed mainly due to hydrophobic interaction and otherwise the membranes behave in a chemically neutral way. Due to the fact that the EPO molecules are located at the surface of the membrane they are easily accessible for antibodies which are used to make the erythropoietin visible. With the help of isoelectrical focusing the isoforms of the erythropoietin can be separated.

For the detection of the erythropoietin the membrane is at first incubated with a monoclonal anti-EPO-antibody which specifically binds to all present EPO molecules. Other proteins do not react with the antibody. Monoclonal antibodies which are not bound to EPO can be washed from the membrane since the rest of the membrane surface has at first been blocked by an unspecific protein.

The binding of the antibody to EPO is reversible since it is based on non-covalent interactions and thus can be reversed for example by means of changes of the pH value. In the double-blotting process the antibodies which are bound to the erythropoietin are transferred onto a second membrane: in an acidic environment the antibody changes the conformation of its binding domain and when an electric field is applied the monoclonal antibody dissociates from the EPO molecule and moves through the electric field in direction of the cathode where it is bound to a second membrane.

The EPO molecules as well as other non-specific proteins remain on the first membrane since the binding to the first membrane is not influenced by fluctuations of the pH value. In this way a new copy of the EPO band or bands is obtained. However there are no erythropoietin molecules on the second membrane but the specific monoclonal antibodies which were previously bound to the erythropoietin molecules fixed on the first membrane.

The antibody band(s) is/are made visible by means of a second antibody (secondary antibody) which reacts with the anti-EPO-monoclonal antibody. This secondary antibody is coupled with special enzymes (e.g. alkaline phosphatase or peroxidase) which catalyse a transformation of the substrate during which a colour reaction occurs.

The second transfer is necessary due to a reduction of unspecific signals since the enzyme-marked secondary antibody can not react unspecifically with other parts of the sample on the first membrane. Furthermore an amplification of the signal and a related increase of the sensitivity by means of multiple bonds of the antibody-enzyme-conjugates to the first antibody is possible. A disadvantage of the double-blotting process is the considerably higher consumption of material and time.

Thus, methods for the detection of erythropoietin are disclosed in the state of the art and are well-known to a person skilled in the art but for the detection of erythropoietin either the considerably more complex double-blotting process is used as described above (Chuan et al., Cytotechnology 2006, 51, 67-79; Hollaender et al., Laborpraxis December 2004, 56-59; Lasne, Journal of Immunological Methods 2003, 276, 223-226) or, when using the electrical focusing process, only a part of the necessary pH range is displayed on the gel so that the selectivity in the separation of the isoforms is not sufficient to evaluate the quality of the EPO raw product (Wimmer et al., Cytotechnology 1994, 16, 137-146).

The methods for the detection of erythropoietin disclosed in the state of the art are, however, too complex and not suitable for an in-process control in the fermentative production of erythropoietin.

The object of the present invention is to offer a simplified and improved method for the detection of erythropoietin which is used in the in-process control and in which the process makes it possible to characterise the culture supernatants from the EPO fermentation processes in such a way that only selected fermentation solutions which have been evaluated as being suitable are introduced into the extensive purification processes. Especially from an economic point of view the present process should be superior to the processes disclosed in the state of the art.

The technical problem is solved by means of a method for determining the isoform composition of erythropoietin comprising the following steps:
a) isoelectrical focusing of a sample comprising erythropoietin in a gel over a pH range having a lower limit of 2.5 to 3.5 and an upper limit of 5 to 8 wherein the sample comprising erythropoietin originates from a culture supernatant of erythropoietin-producing eukaryotic cells;
b) transferring the proteins comprised and separated in the gel to a membrane;
c) verifying the erythropoietin bound to the membrane by specific antibodies.

In a preferred method first antibodies which are directed against erythropoietin are bound in step c) to the erythropoietin which is bound to the membrane wherein the binding of these first antibodies to the erythropoietin which is bound to the membrane is detected while the first antibody is bound to the erythropoietin which is bound to the membrane.

The advantage is that the binding of the first antibodies to the erythropoietin which is bound to the membrane is detected by means of second antibodies which are directed against the first antibodies.

This means that in the process according to step c) first antibodies which are directed against erythropoietin bind to the erythropoietin which is bound to the membrane and that second antibodies which are directed against the first antibodies bind to the first antibodies which are bound to erythropoietin.

Due to the fact that, additionally to the determination of the EPO content, preferably by means of ELISA, the isoform composition of the fermentation supernatants are directly determined with the help of a special isoelectrical focusing process (IEF) in combination with a single blotting step an advantageous and simplified method is provided which can be used to control the fermentation process and to decide upon the selection of the culture supernatants which must be purified.

In view of the present state of the art the person skilled in the art, being confronted with the object as mentioned above, would not have considered with the hope to succeed that the process according to the invention can be used for the selection of fermentation fractions before the purification process. So far a comparably simple and efficient method has not been described in literature. It is particularly advantageous that with the isoelectrical focusing process according to the invention a pH range having a lower limit of 2.5 to 3.5 and an upper limit of 5 to 8, especially a pH range from 3 to 6, is displayed on the gel so that the necessary selectivity in the separation of the isoforms is sufficient to assess the quality of the EPO raw product. Further, in a preferred embodiment the process is simplified to such a degree that the erythropoietin can already be detected after a single protein transfer (blot). In the state of the art, in contrast, the significantly more complex double-blotting process is used for the detection.

In a preferred method an enzyme, preferably alkaline phosphatase, is covalently bound to the second antibody which causes a reaction of colour by means of the catalytic reaction of a substrate. Thus, for the colorimetric detection of EPO it is preferred to apply two antibody solutions to the membrane, the second antibody containing an alkaline phosphatase, so that later a substrate of said enzyme can be used as colourant reagent. Especially preferred is the use of anti-EPO-mouse and anti-mouse-IgG in combination with BCIP/NPT (5-bromo-4-chloro-3-indolylphosphate/nitrotetrazolium blue).

Furthermore it is preferred that after the incubation of the membrane with a first antibody which is directed against erythropoietin one or more steps of washing are performed and subsequently a further incubation of the membrane with the first antibody which is directed against erythropoietin is carried out. This double or multiple incubation of the membrane with the antibody which is directed against erythropoietin makes a more complete development of the antigen-antibody-reaction possible and thus an increase of the specific signal for erythropoietin.

In an especially preferred method at least one solution used during the steps of washing which follow an incubation of the membrane with the first antibody which is directed against erythropoietin contains an organic acid in an aqueous medium. In this way unspecifically bound antibodies are again removed from the membrane and possible free bonding sites on the blotting membrane are blocked. All in all this considerably increases the selectivity of the bands. It is preferred that the solution contains 0.1 to 1.5% by weight, preferably 0.5 to 1%) by weight, more preferably 0.6 to 0.8% by weight of the organic acid. In particular the organic acid can be mono-, di- or tricarboxylic acids (such as acetic acid, propanoic acid, lactic acid, succinic acid, ascorbic acid, adipic acid or citric acid), especially preferred acetic acid.

As mentioned above in one preferred method the incubation with the first antibody solution is carried out twice and in between a washing procedure comprising four steps is carried out. Here, preferably TBST (Tris-Buffered-Saline-Tween) and TBS (Tris-Buffered-Saline), a diluted, aqueous, organic acid, is used. Especially preferably a diluted, aqueous acetic acid is used and even more preferably a diluted, aqueous acetic acid within a concentration range of between 0.5% and 1% is used.

In one preferred method a poly(vinylidene fluoride) membrane is used as a membrane. In a preferred way a membrane is used for the blotting which is suitable to bind proteins. Especially preferred is a microporous poly(vinylidene fluoride) membrane (PVDF) and even more preferred is the Immobilon-P-blotting membrane of Millipore Company.

In another preferred method polyacrylamide gels, which are applied to an inert carrier foil, are used for the isoelectrical focusing process. Preferably, standardised polyacrylamide gels, which are bound to an inert carrier foil, e.g. made of polyester, are used for the IEF. Especially preferred are gels whose pH gradient is formed in the electric field by means of carrier ampholytes. Even more preferred are Blank PreNets of the company Serva.

In particular it is preferred that to adjust the pH value of the gel ampholines are used which adjust a pH range from pH 3 to pH 6 in the gel during isoelectrical focusing. Especially preferred is the use of Servalyt™ 3-6 of the company Serva.

In a further preferred method the erythropoietin-comprising sample originates from a culture supernatant of erythropoietin-producing eukaryotic cells grown in perfusion reactors. Preferably the erythropoietin-comprising sample is desalinated and if required concentrated before the isoelectrical focusing process.

In an especially preferred embodiment of the method according to the invention the isoform composition of the erythropoietin is determined during fermentation.

As well preferred is the use of an ELISA test in order to determine the EPO content of the culture supernatants. Especially preferred is the EPO ELISA test of the company Roche Diagnostics.

The method according to the invention results in an EPO production process which requires considerably fewer machines and human resources, consequently translating into a significant saving of time and costs. Not later than 24 hours after obtaining the sample of a culture supernatant, e.g. from a perfusion fermentation process, it can be decided whether a purification of the fermentation solution makes sense and how the purification process can be controlled.

A commercially available Erypo® preparation (Janssen-Cilag) serves as reference material.

The invention furthermore provides a method for in-process control of culture supernatants which originate from the fermentation of erythropoietin-producing eukaryotic cells comprising the following steps:

a) determining the isoform composition of erythropoietin according to the method for detection of erythropoietin according to the invention as described above;
b) determining the content of erythropoietin in the sample, preferably by means of ELISA;
c) selection of the fermentation solutions for the purification of erythropoietin with the help of the values and information obtained in steps a) and b), or continuation of the fermentation.

The method is especially characterised in that the isoform composition of the fermentation supernatants can be directly determined with the help of a special isoelectrical focusing process (IEF). Together with the data obtained from the determination of the EPO content (preferably by means of ELISA) the EPO quality in the raw product can already be evaluated during or directly upon completion of the fermentation process and thus the subsequent purification process can be controlled.

The following example is meant to explain the invention without limiting the scope of it.

### DESCRIPTION OF THE FIGURE

FIG. 1 shows a blot of different EPO fractions (fraction 1 to fraction 5) on a membrane after isoelectrical focusing and development. The sample originates from the perfusion fermentation of an erythropoietin-producing CHO cell line over a period of 47 days.

### EXAMPLE

EPO is fermentatively produced in CHO cells. The fermentation is carried out with the help of standardised procedures as described for eukaryotic cells, in particular CHO cells, in patent and scientific literature. The cultivation takes place in the perfusion reactor in a culture medium which does not contain any animal components. The harvest takes place continuously within a time period of up to 50 days.

Each fermentation solution which has to be analysed is desalinated and concentrated before the isoelectrical focusing process. To this end at first 15 mL of the sample are compressed to 200 μL with the help of the ultra-centrifugation kit and a molecular weight Cut Off of 10 kDa by means of centrifugation (60 min with 4000 g and 60 min with 14000 g) at a total EPO content of about 14 mg/L (determined by the ELISA test) and 12 μL of the concentrate is mixed with 28 μL of ultrapure water and 10 μL of ethanol and is stored for 60 min at −20° C. Afterwards the sample solution is centrifuged in a refrigerated centrifuge (20 min, 16100 g, 0° C.) and the supernatant of the solution is used for isoelectrical focusing (IEF sample solution).

The isoelectrical focusing process starts with prefocusing the gel (Blank PreNets, Serva; 20 to 60 min up to approx. 400 Vh) in order to develop the pH gradient from pH 3 to pH6 (Servalyt™ 3-6). To this end a voltage value of approx. 300V and a current value of 3.5 mA is chosen (cathode buffer: 1 M glycine; anode buffer: 25 mM of aspartic acid and glutamic acid, respectively). After that 15 μL of the IEF sample solution and the control solution (Erypo®-preparation), respectively, are pipetted on a Sample Application Piece (Serva) and the solutions are isoelectrically focused at approx. 2000 Vh by applying voltage. Next, the focusing process is shortly interrupted and the Sample Application Pieces are removed before the focusing process is continued at further 2500 Vh. After exceeding a sample focusing time of 4500 Vh the isoelectrical focusing process is stopped and the gel is incubated for 15 min in a precooled (4° C.) blotting buffer (200 mL 10× Tris/Glycine Buffer (Bio-Rad) are diluted with ultrapure water and 400 mL of methanol to 2 L).

In a parallel step the Immobilon-P-blotting membrane (Millipore) is prepared according to the instructions of the supplier. Afterwards the gel is blotted onto the membrane under the following conditions: 50V constantly for 50 min in the blotting buffer (1× Tris/Glycine with 20% of methanol, Bio-Rad). Directly after the protein transfer three washing steps are carried out one after the other, first in methanol and then twice in water for 30 seconds, respectively. Subsequently the membrane is put into a blocking solution (a 5% skimmed milk powder solution (Bio-Rad) in 1×TBS buffer (Bio-Rad)) and is incubated at room temperature for 60 min while subjected to gentle shaking. After removing the blocking solution it is washed in TBST (0.5% Tween® 20 in TBS (Bio-Rad)) three times. Afterwards an incubation of at least 4 hours in the first antibody solution takes place (1% BSA (Sigma) in 30 ml 1×TBS buffer (Bio-Rad) with 60 μl 500 mM sodium azide solution of the 50 μl Anti-EPO-Mouse (RD-Systems)).

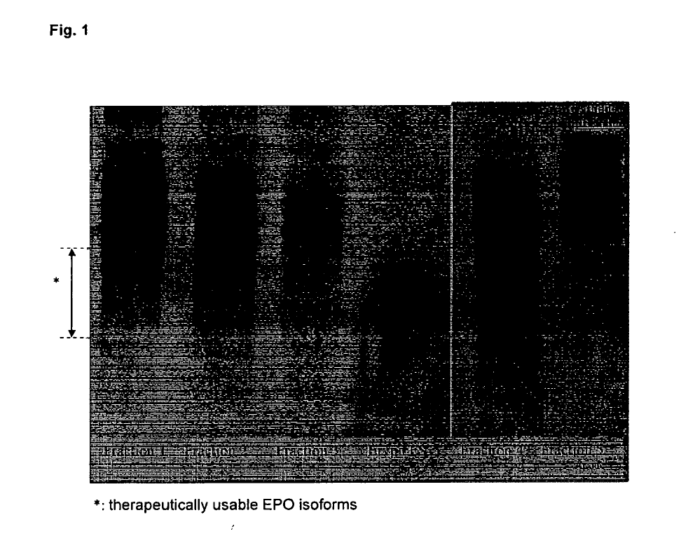

Then a further washing procedure with TBST, TBS, 0.7% aqueous acetic acid and again TBST follows wherein the membrane is incubated for 60 min in the acetic acid solution. Then the treatment of the membrane is repeated with the first antibody solution under identical conditions. After washing it three times with TBST the treatment of the membrane with the second antibody solution takes place (1% BSA (Sigma) in 30 ml 1×TBS buffer (Bio-Rad) with 60 μl 500 mM sodium azide to which 35 μl of Anti-Mouse-IgG (Sigma) is added). After washing it three times in TBST and rinsing it twice with AP buffer (10 mL 5M saline solution is diluted with 50 mL 1 M Tris-HCl-solution (pH 9.5) and 5 mL 1 M magnesium chloride solution and ultrapure water to a solution volume of 1 L) the gel is coloured with BCIP/NBT Liquid Substrate System (Sigma) (10 to 20 min at room temperature while subjecting it to gentle shaking). By adding AP stop solution (10 ml 0.5 M Na-EDTA solution (pH 8.0) is diluted with 20 mL 1 M Tris-HCl solution (pH 8.0) and ultrapure water to a solution volume of 1 L) the reaction of colour is stopped and the membrane is rinsed again with ultrapure water. After air drying the membrane can be evaluated visually or densitometrically (see FIG. 1).

The EPO content of the culture supernatants is determined by means of the EPO ELISA Test of the company Roche Diagnostics GmbH (photometric enzyme-bound Immuno Sorbent Assay for the quantitative in vitro determination of erythropoietin in human serum/plasma for research purposes by using antibody-precoated microtiter plates).

For the single fractions from the perfusion reactor (total fermentation time of 47 days) EPO contents in the following ranges are calculated:

Fraction 1 (fermentation up to day 5): approx. 80 mg/L
Fraction 2 (fermentation up to day 13): approx. 75 mg/L
Fraction 3 (fermentation up to day 20): approx. 140 mg/L
Fraction 4 (fermentation up to day 25): approx. 80 mg/L
Fraction 5 (fermentation up to day 32): approx. 150 mg/L

The evaluation of the results according to FIG. 1 shows that particularly the purification of Fraction 2 and 4 and probably also Fraction 3 makes sense. These fractions have the highest percentage of therapeutically usable isoforms in relation to the total EPO content. By contrast, Fraction 5 has the highest EPO content according to the ELISA test however the desired isoforms compared to the Erypo® reference material are only contained in very traces.

1. Method for the in-process control of culture supernatants of the fermentation of erythropoietin-producing eukaryotic cells characterised by the following steps:
a) obtaining a erythropoietin-comprising sample from a culture supernatant of erythropoietin-producing eukaryotic cells;
b) isoelectrical focusing of an erythropoietin-containing sample in a gel over a pH range having a lower limit of 2.5 to 3.5 and an upper limit of 5 to 8;
c) transferring the proteins comprised and separated in the gel to a membrane;
d) detection of the erythropoietin bound to the membrane by means of specific antibodies wherein the isoform composition of the erythropoietin is obtained;
e) determining the content of erythropoietin in the sample, preferably with the help of ELISA; and
f) if required purifying the fermentation solution;
wherein in step d) one or more washing steps are carried out after the incubation of the membrane with a first antibody which is directed against erythropoietin and subsequently another incubation of the membrane with the first antibody which is directed against erythropoietin is carried out wherein at least one solution used during the steps of washing which follow an incubation of the membrane with the first antibody which is directed against erythropoietin contains an organic acid in an aqueous medium.

2. Method according to claim 1 wherein first antibodies which are directed against erythropoietin bind in step d) to the erythropoietin which is bound to the membrane and that the binding of these first antibodies to the erythropoietin which is bound to the membrane is detected while the first antibody is bound to the erythropoietin which is bound to the membrane.

3. Method according to claim 2 wherein the binding of the first antibodies to the erythropoietin which is bound to the membrane is detected by means of second antibodies which are directed against the first antibodies.

4. Method according to claim 1 wherein in step d) first antibodies which are directed against erythropoietin bind to the erythropoietin which is bound to the membrane and that second antibodies which are directed against the first antibodies bind to the first antibodies which are bound to erythropoietin.

5. Method according to claim 3 wherein an enzyme, preferably alkaline phosphatase, is covalently bound to the second antibody which causes a reaction of colour by means of the catalytic reaction of a substrate.

6-7. (canceled)

8. Method according to claim 1 wherein the solution contains 0.1 to 1.5% by weight, preferably 0.5 to 1% by weight, more preferably 0.6 to 0.8% by weight of the organic acid.

9. Method according to claim 8 wherein the organic acid is a mono-, di- or tricarboxylic acid, preferably selected from the group consisting of acetic acid, propanoic acid, lactic acid, succinic acid, ascorbic acid, adipic acid or citric acid, and especially preferred is acetic acid.

10. Method according to claim 1 wherein a poly(vinylidene fluoride) membrane is used as a membrane.

11. Method according to claim 1 wherein polyacrylamide gels, which are applied to an inert carrier foil, are used for the isoelectrical focusing process.

12. Method according to claim 1 wherein to adjust the pH value of the gel ampholines are used which adjust a pH range from pH 3 to pH 6 in the gel during isoelectrical focusing.

13. Method according to claim 1 wherein the erythropoietin-comprising sample originates from a culture supernatant of erythropoietin-producing eukaryotic cells grown in perfusion reactors.

14. Method according to claim 13 wherein the erythropoietin-comprising sample is desalinated and if required concentrated before the isoelectrical focusing process.

15. Method according to claim 1 wherein the isoform composition of the erythropoietin is determined during fermentation.

16. (canceled)
